(19) United States
(12) Patent Application Publication
(10) Pub. No.: US 2022/0325321 A1
(43) Pub. Date: Oct. 13, 2022

(54) Intestinal microflora and its combination as biomarkers and detection methods for liver cancer

(71) Applicant: National Yang Ming Chiao Tung University, Taipei City (TW)

(72) Inventors: Chun-Ying WU, Taipei City (TW); Jiunn-Chang LIN, New Taipei City (TW)

(21) Appl. No.: 17/235,431

(22) Filed: Apr. 20, 2021

(30) Foreign Application Priority Data
Apr. 13, 2021 (CN) 202110394539.X

(51) Int. Cl. C12Q 1/689; G01N 33/574

(52) U.S. Cl. CPC C12Q 1/689; G01N 33/57438; C12Q 2600/158

(57) ABSTRACT

The invention uses intestinal microbial flora and combinations thereof as a liver cancer biomarker and provide a method for detection, in short, the intestinal microbial flora of the invention can be used as a liver cancer biomarker and can be used as a non-invasive method for detecting or predicting liver cancer, combined with conventional blood tests, not only capable of improving the accuracy rate of detection, but also capable of increasing the willingness of patients to participate in detection to achieve the efficacies of early prevention and early treatment.

### BACKGROUND OF THE INVENTION

### Field of Invention

The invention relates to a method for detecting cancer and a biomarker used, and more particularly to using intestinal microbial flora and combinations thereof as a liver cancer biomarker and a method for detection.

### Related Art

Liver cancer is one of the top ten cancers in Taiwan, China. Since liver cancer does not cause pain in the early stage, most patients already have advanced liver cancer when it is detected, and treatment is extremely difficult, so not only the incidence of liver cancer is high, but the fatality rate has always been high, which has a great impact on the health of Taiwanese people and the country's economic losses. Although studies have pointed out that the risk factors of liver cancer include chronic hepatitis, alcoholism, long-term use of aflatoxin, and fatty liver, how to effectively track hepatitis patients and detect the risk of liver cancer in early stage is still a major problem in clinical medicine.

Currently, alpha-fetoprotein (AFP) is used as a biomarker to detect liver cancer in clinical practice. However, even if the abnormality of AFP is detected clinically, it can only represent that the liver is in an inflamed state, and cannot correctly detect whether the patient has liver cancer. Therefore, for the high-risk groups of liver cancer, it is still insufficient to rely on the test results of AFP to determine whether the person has liver cancer.

In recent years, the role of intestinal microbial flora in disease detection has gradually received attention. The United States National Institutes of Health (NIH) initiated the Human Microbiome Project in 2008, which is mainly to understand the functions of microorganisms (human microbiome) related to human health and disease. However, due to the large number and complexity of intestinal microbes, considering the detection costs and detection accuracy rate, how to select the appropriate intestinal microbes as disease biomarkers is a big problem.

### SUMMARY OF THE INVENTION

A main object of the invention is to use intestinal microbial flora and combinations thereof as a liver cancer biomarker and provide a method for detection, which is a model established by combining the intestinal microbial flora with the current liver cancer biomarker alpha-fetoprotein (AFP) in order to achieve efficacies of enhancing sensitivity and specificity higher than detection of a single AFP biomarker and improving diagnosis and prediction accuracy rate of liver cancer.

Another object of the invention is to use intestinal microbial flora and combinations thereof as a liver cancer biomarker and provide a method for detection capable of detecting liver cancer in a non-invasive manner and combined with current conventional blood tests, which can not only increase the willingness of patients to participate in tests, but also achieve efficacies of early detection and early treatment of liver cancer, and reducing the burdens on the medical system's manpower and social economy.

Therefore, in order to achieve the above objects, the invention provides a method for increasing an accuracy rate of detecting cancer or predicting cancer risk in vitro, which comprises simultaneously detecting and analyzing manifestations of at least one microorganism in a stool sample and alpha-fetoprotein (AFP) content in a blood sample, wherein:

the microbial genus is selected from a group consisting of Lachnospira, Succinivibrio, Phascolarctobacterium, rc4_4, Sutterella, WAL_1855D, Methanobrevibacter, Bacteroides, Megasphaera, Dialister, Streptococcus, Acidaminococcus, Epulopiscium, Lactobacillus, Enterococcus, Eubacterium, Rothia, Bifidobacterium, Collinsella, Clostridium, Leuconostoc, Ruminococcus, Rhodococcus, Catenibacterium, Mitsuokella, Roseburia, Granulicatella, Citrobacter, Lactococcus, Coprobacillus, Blautia, Klebsiella, Helicobacter, Succiniclasticum, Peptococcus, Megamonas, Peptostreptococcus, Veillonella, Salmonella, Akkermansia, Corynebacterium and Faecalibacterium.

Preferably, at least the following five bacterial genera are comprised in detection and analysis of microbial manifestations in the stool sample: Phascolarctobacterium, Lachnospira, Megasphaera, Streptococcus and Bifidobacterium.

Preferably, the cancer is liver cancer.

The invention provides a method for increasing an accuracy rate of detecting liver cancer risk in vitro by simultaneously detecting and analyzing manifestations of at least one microorganism in a stool sample and alpha-fetoprotein (AFP) content in a blood sample, wherein:

the microbial species is selected from a group consisting of Akkermansia muciniphila, Faecalibacterium prausnitzii, Bacteroides barnesiae, Eubacterium biforme, Haemophilus parainfluenzae, Veillonella dispar, Roseburia faecis, Bifidobacterium longum, Collinsella aerofaciens, Lactobacillus mucosae, Helicobacter pylori, Ruminococcus gnavus, Eubacterium dolichum, Rothia mucilaginosa, Mitsuokella multacida, Collinsella stercoris, Lactobacillus zeae, Coprobacillus cateniformis and Rothia aeria.

Preferably, at least the following four bacterial species are comprised in detection and analysis of microbial manifestations in the stool sample: Bifidobacterium longum, Ruminococcus gnavus, Eubacterium dolichum and Rothia mucilaginosa.

The invention provides a biomarker for auxiliary diagnosis and detection of liver cancer, which comprises at least one microorganism, and the microorganism comes from the following bacterial genera: Lachnospira, Succinivibrio, Phascolarctobacterium, rc4_4, Sutterella, WAL_1855D, Methanobrevibacter, Bacteroides, Megasphaera, Dialister, Streptococcus, Acidaminococcus, Epulopiscium, Lactobacillus, Enterococcus, Eubacterium, Rothia, Bifidobacterium, Collinsella, Clostridium, Leuconostoc, Ruminococcus, Rhodococcus, Catenibacterium, Mitsuokella, Roseburia, Granulicatella, Citrobacter, Lactococcus, Coprobacillus, Blautia, Klebsiella, Helicobacter, Succiniclasticum, Peptococcus, Megamonas, Peptostreptococcus, Veillonella, Salmonella, Akkermansia, Corynebacterium and Faecalibacterium; or is obtained from the following bacterial species: Akkermansia muciniphila, Faecalibacterium prausnitzii, Bacteroides barnesiae, Eubacterium biforme, Haemophilus parainfluenzae, Veillonella dispar, Roseburia faecis, Bifidobacterium longum, Collinsella aerofaciens, Lactobacillus mucosae, Helicobacter pylori, Ruminococcus gnavus, Eubacterium dolichum, Rothia mucilaginosa, Mitsuokella multacida, Collinsella stercoris, Lactobacillus zeae, Coprobacillus cateniformis and Rothia aeria.

In one embodiment of the invention, the biomarker comprises a plurality of microorganisms, and the microorganisms respectively come from the following bacterial genera: Phascolarctobacterium, Lachnospira, Megasphaera, Streptococcus and Bifidobacterium.

In another embodiment of the invention, the biomarker comprises a plurality of microorganisms, and the microorganisms respectively come from the following bacterial species: Bifidobacterium longum, Ruminococcus gnavus, Eubacterium dolichum and Rothia mucilaginosa.

In one embodiment of the invention, the invention provides a secondary use of an intestinal flora analysis kit, which can be used to assist in predicting liver cancer risk, specifically, the intestinal flora analysis kit is capable of detecting manifestations of at least one microorganism in a stool sample, and the microorganism comes from the following groups of bacterial genera: Lachnospira, Succinivibrio, Phascolarctobacterium, rc4_4, Sutterella, WAL_1855D, Methanobrevibacter, Bacteroides, Megasphaera, Dialister, Streptococcus, Acidaminococcus, Epulopiscium, Lactobacillus, Enterococcus, Eubacterium, Rothia, Bifidobacterium, Collinsella, Clostridium, Leuconostoc, Rumninococcus, Rhodococcus, Catenibacterium, Mitsuokella, Roseburia, Granulicatella, Citrobacter, Lactococcus, Coprobacillus, Blautia, Klebsiella, Helicobacter, Succiniclasticum, Peptococcus, Megamonas, Peptostreptococcus, Veillonella, Salmonella, Akkermansia, Corynebacterium and Faecalibacterium; or comes from the following groups of bacterial species: Akkermansia muciniphila, Faecalibacterium prausnitzii, Bacteroides barnesiae, Eubacterium biforme, Haemophilus parainfluenzae, Veillonella dispar, Roseburia faecis, Bifidobacterium longum, Collinsella aerofaciens, Lactobacillus mucosae, Helicobacter pylori, Ruminococcus gnavus, Eubacterium dolichum, Rothia mucilaginosa, Mitsuokella multacida, Collinsella stercoris, Lactobacillus zeae, Coprobacillus cateniformis and Rothia aeria.

Wherein, the intestinal flora analysis kit is a well-known kit for those skilled in the art to which the invention belongs. Specifically, the intestinal flora analysis kit comprises extracting DNA of microorganisms in the stool sample and identifying the microorganisms with 16S next-generation sequencing method to obtain the intestinal flora of the stool sample provider.

Preferably, the microorganisms respectively come from the following bacterial genera: Phascolarctobacterium, Lachnospira, Megasphaera, Streptococcus and Bifidobacterium.

Preferably, the microorganisms respectively come from the following bacterial species: Bifidobacterium longum, Ruminococcus gnavus, Eubacterium dolichum and Rothia mucilaginosa.

The beneficial effects of the invention are:

the biomarker of the invention can be used to detect the occurrence of liver cancer or predict the risk of occurrence of liver cancer in vitro, specifically, one embodiment of the invention provides a method for detecting liver cancer in vitro, which simultaneously detecting and analyzing manifestations of the biomarker in a stool sample and alpha-fetoprotein (AFP) content in a blood sample, and through the test results capable of determining the risk and possibility of liver cancer in the stool sample and blood sample provider; and not only capable of improving the detection accuracy rate, but also capable of increasing the willingness of patients to participate in detection to achieve the efficacies of early prevention and early treatment.

### BRIEF DESCRIPTION OF THE DRAWINGS

FIG. 1 shows relative content distribution of bacterial genera with significant differences between liver cancer patients and healthy individuals;

FIG. 2 shows relative content distribution of bacterial species with significant differences between liver cancer patients and healthy individuals;

FIG. 3 shows the ROC curve analysis results of MDI conversion results (MDI), alpha-fetoprotein (AFP) of 42 bacterial genera, and MDI conversion results combined with alpha-fetoprotein (MDI+AFP) of 42 bacterial genera;

FIG. 4 shows the ROC curve analysis results of MDI conversion results (MDI), alpha-fetoprotein (AFP) of 19 bacterial species, and MDI conversion results combined with alpha-fetoprotein (MDI+AFP) of 19 bacterial species;

FIG. 5 shows the ROC curve analysis results of genus Lachnospira (MDI), alpha-fetoprotein (AFP), and genus Lachnospira combined with alpha-fetoprotein (MDI+AFP);

FIG. 6 shows the ROC curve analysis results of genus Phascolarctobacterium (MDI), alpha-fetoprotein (AFP), and genus Phascolarctobacterium combined with alpha-fetoprotein (MDI+AFP);

FIG. 7 shows the ROC curve analysis results of genus Megasphaera (MDI), alpha-fetoprotein (AFP), and genus Megasphaera combined with alpha-fetoprotein (MDI+AFP);

FIG. 8 shows the ROC curve analysis results of genus Dialister (MDI), alpha-fetoprotein (AFP), and genus Dialister combined with alpha-fetoprotein (MDI+AFP);

FIG. 9 shows the ROC curve analysis results of genus Streptococcus (MDI), alpha-fetoprotein (AFP), and genus Streptococcus combined with alpha-fetoprotein (MDI+AFP);

FIG. 10 shows the ROC curve analysis results of genus Acidaminococcus (MDI), alpha-fetoprotein (AFP), and genus Acidaminococcus combined with alpha-fetoprotein (MDI+AFP);

FIG. 11 shows the ROC curve analysis results of genus Lactobacillus (MDI), alpha-fetoprotein (AFP), and genus Lactobacillus combined with alpha-fetoprotein (MDI+AFP);

FIG. 12 shows the ROC curve analysis results of genus Enterococcus (MDI), alpha-fetoprotein (AFP), and genus Enterococcus combined with alpha-fetoprotein (MDI+AFP);

FIG. 13 shows the ROC curve analysis results of genus Eubacterium (MDI), alpha-fetoprotein (AFP), and genus Eubacterium combined with alpha-fetoprotein (MDI+AFP);

FIG. 14 shows the ROC curve analysis results of genus Bifidobacterium (MDI), alpha-fetoprotein (AFP), and genus Bifidobacterium combined with alpha-fetoprotein (MDI+AFP);

FIG. 15 shows the ROC curve analysis results of genus Collinsella (MDI), alpha-fetoprotein (AFP), and genus Collinsella combined with alpha-fetoprotein (MDI+AFP);

FIG. 16 shows the ROC curve analysis results of genus Clostridium (MDI), alpha-fetoprotein (AFP), and genus Clostridium combined with alpha-fetoprotein (MDI+AFP);

FIG. 17 shows the ROC curve analysis results of five bacterial genera of Phascolarctobacterium, Lachnospira, Megasphaera, Streptococcus and Bifidobacterium (MDI), alpha-fetoprotein (AFP), and the aforementioned five bacterial genera combined with alpha-fetoprotein (MDI+AFP);

FIG. 18 shows relative content distribution of five bacterial genera of Phascolarctobacterium, Lachnospira, Megasphaera, Streptococcus and Bifidobacterium;

FIG. 19 shows the ROC curve analysis results of species Ruminococcus gnavus (MDI), alpha-fetoprotein (AFP), and species Ruminococcus gnavus combined with alpha-fetoprotein (MDI+AFP);

FIG. 20 shows the ROC curve analysis results of species Bifidobacterium longum (MDI), alpha-fetoprotein (AFP), and species Bifidobacterium longum combined with alpha-fetoprotein (MDI+AFP);

FIG. 21 shows the ROC curve analysis results of species Rothia mucilaginosa (MDI), alpha-fetoprotein (AFP), and species Rothia mucilaginosa combined with alpha-fetoprotein (MDI+AFP);

FIG. 22 shows the ROC curve analysis results of species Collinsella aerofaciens (MDI), alpha-fetoprotein (AFP), and species Collinsella aerofaciens combined with alpha-fetoprotein (MDI+AFP);

FIG. 23 shows the ROC curve analysis results of species Eubacterium dolichum (MDI), alpha-fetoprotein (AFP), and species Eubacterium dolichum combined with alpha-fetoprotein (MDI+AFP);

FIG. 24 shows the ROC curve analysis results of four bacterial species of Bifidobacterium longum, Ruminococcus gnavus, Eubacterium dolichum and Rothia mucilaginosa (MDI), alpha-fetoprotein (AFP), and the aforementioned four bacterial species combined with alpha-fetoprotein (MDI+AFP); and

FIG. 25 shows relative content distribution of four bacterial species of Bifidobacterium longum, Ruminococcus gnavus, Eubacterium dolichum and Rothia mucilaginosa.

### DETAILED DESCRIPTION OF THE INVENTION

The objects, features, and achieved efficacies of the invention can be understood from the description and drawings of the following preferred embodiments.

The “MDI” referred to in the following embodiments is the abbreviation of microbial dysbiosis index, which uses LEfSe (Linear discriminant analysis Effect Size) analysis method to pick out significant bacterial genus and bacterial species-level flora. After the flora is grouped, the following formula (I) is used to calculate the MDI of the specimen, and calculation of the probability of an estimated case is shown in the following formula (II).

$$M D {I}_{i} = log ( \frac{{\sum }_{j ∈ case} {p}_{ij}}{{\sum }_{i ∈ ctrl} {p}_{ij}} ) \qquad ( I )$$

$$prob ( case \vert M D I = x ) = \frac{den ( M D I = x \vert case )}{den ( M D I = x \vert case ) = den ( M D I = x \vert ctrl )} \qquad ( II )$$

### Embodiment 1: Analysis of Microbial Flora (1)

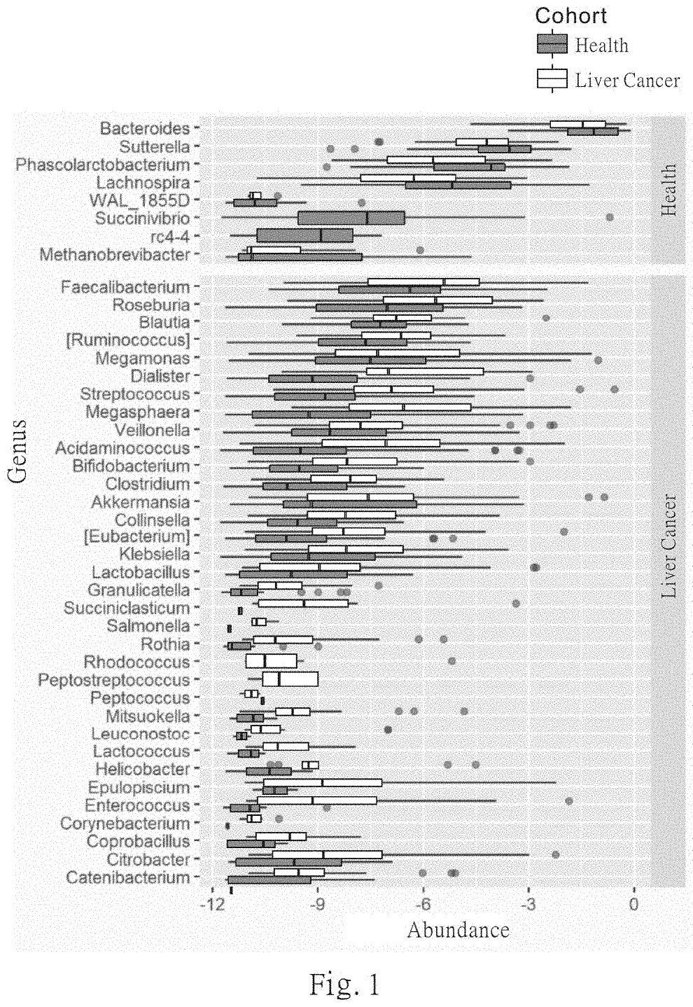

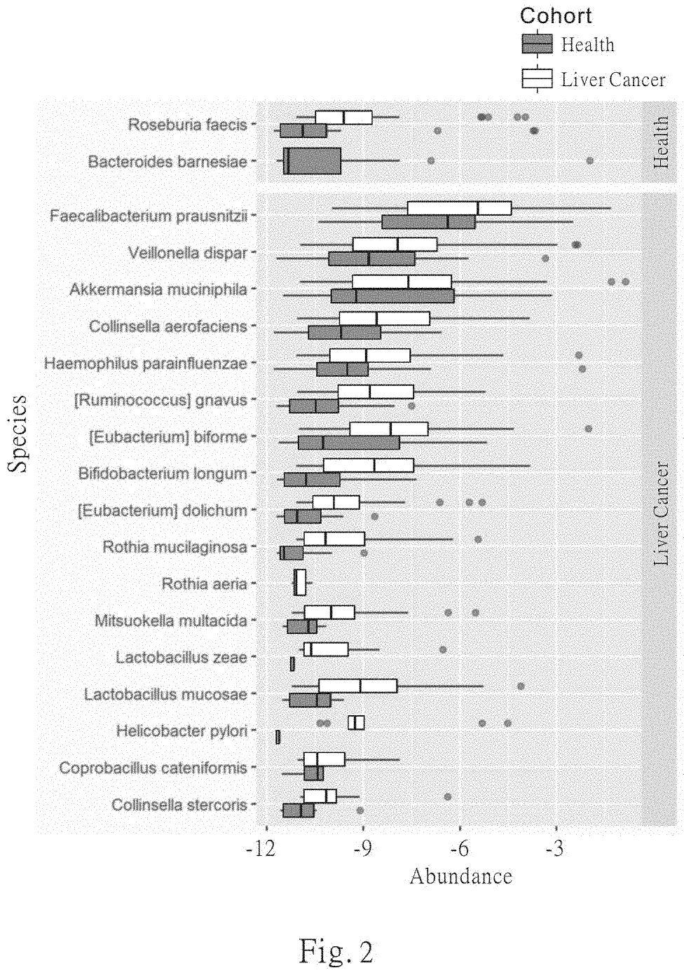

As shown in table 1, the paired liver cancer patients and healthy individuals are used as two groups of participants. DNA from their stool samples is extracted respectively, the intestinal flora is identified by the 16S next-generation sequencing method, and the LEfSe analysis method is used to analyze the results of the gastrointestinal flora of the two groups of participants, it can be known that the gastrointestinal flora in the stool samples provided by the two groups of participants are different as shown in FIGS. 1 and 2, wherein:

as can be known from FIG. 1, there are significant differences in the distribution of the following forty-two bacterial genera between the two groups of participants: Lachnospira, Succinivibrio, Phascolarctobacterium, rc4_4, Sutterella, WAL_1855D, Methanobrevibacter, Bacteroides, Megasphaera, Dialister, Streptococcus, Acidaminococcus, Epulopiscium, Lactobacillus, Enterococcus, Eubacterium, Rothia, Bifidobacterium, Collinsella, Clostridium, Leuconostoc, Ruminococcus, Rhodococcus, Catenibacterium, Mitsuokella, Roseburia, Granulicatella, Citrobacter, Lactococcus, Coprobacillus, Blautia, Klebsiella, Helicobacter, Succiniclasticum, Peptococcus, Megamonas, Peptostreptococcus, Veillonella, Salmonella, Akkermansia, Corynebacterium and Faecalibacterium;

as can be known from FIG. 2, there are significant differences in the distribution of the following nineteen bacterial species between the two groups of participants: Akkermansia muciniphila, Faecalibacterium prausnitzii, Bacteroides barnesiae, Eubacterium biforme, Haemophilus parainfluenzae, Veillonella dispar, Roseburia faecis, Bifidobacterium longum, Collinsella aerofaciens, Lactobacillus mucosae, Helicobacter pylori, Ruminococcus gnavus, Eubacterium dolichum, Rothia mucilaginosa, Mitsuokella multacida, Collinsella stercoris, Lactobacillus zeae, Coprobacillus cateniformis and Rothia aena.

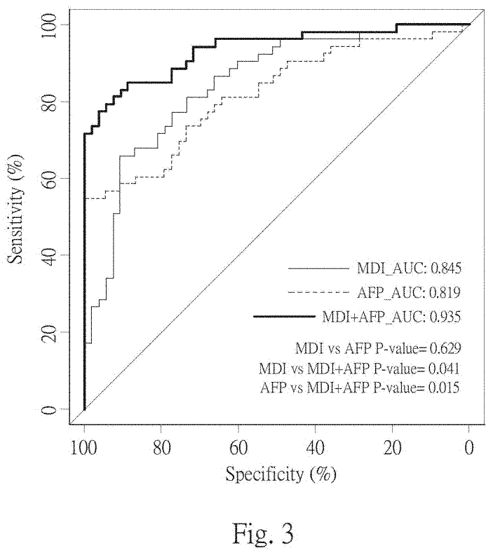

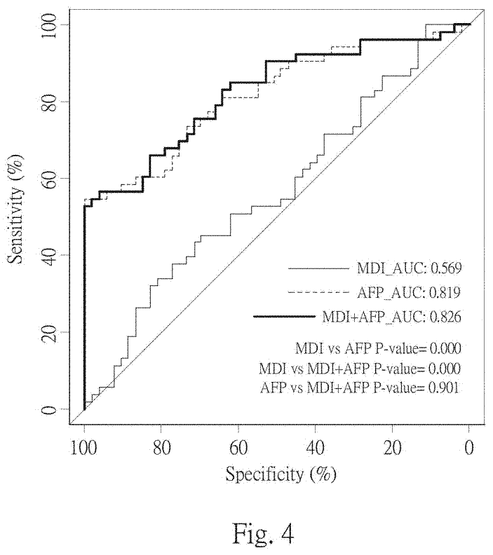

Further, the results of the above forty-two bacterial genera and nineteen bacterial species are converted by MDI (microbial dysbiosis index), and then the above MD conversion results, alpha-fetoprotein (AFP), and the combination of MDI and AFP are analyzed by ROC curves, and comparing the AUC of MDI, AFP and MDI+AFP to obtain the results shown in FIG. 3 and FIG. 4.

From the results in FIG. 3, it can be known that the classification correct rate of liver cancer patients is only about 80% by detecting alpha-fetoprotein (AFP) alone, and when alpha-fetoprotein (AFP) is combined with MDI of the forty-two bacterial genera, the correct rate of detecting liver cancer can be significantly improved to about 94%; and the results in FIG. 4 show that when alpha-fetoprotein (AFP) is combined with MDI of the nineteen bacterial species, the correct rate of detecting occurrence of liver cancer can also be improved.

TABLE 1
Two groups of participants after pairing

| | | | | Liver | | |
|---|---|---|---|---|---|---|
| | Status | Healthy | | cancer | | P |
| Variable | [stat] | individuals | | patients | | value |
| cohort | | 53 | | 53 | | NA |
| Gender | Female | 17 | (32.1%) | 12 | (22.6%) | 0.383 |
| Gender | Male | 36 | (67.9%) | 41 | (77.4%) | 0.383 |
| Stage of | Healthy | 53 | (100.0%) | NA | | <.001 |
| liver | IA | NA | | 12 | (22.6%) | <.001 |
| cancer | IB | NA | | 15 | (28.3%) | <.001 |
| | II | NA | | 15 | (28.3%) | <.001 |
| | IIIA | NA | | 1 | (1.9%) | <.001 |
| | IIIB | NA | | 10 | (18.9%) | <.001 |
| Stage of | Healthy | 53 | (100.0%) | NA | | <.001 |
| liver | I | NA | | 27 | (50.9%) | <.001 |
| cancer-1 | II | NA | | 15 | (28.3%) | <.001 |
| | III | NA | | 11 | (20.8%) | <.001 |
| Stage of | Healthy | 53 | (100.0%) | NA | | <.001 |
| liver | Early | NA | | 27 | (50.9%) | <.001 |
| cancer-2 | Advanced | NA | | 26 | (49.1%) | <.001 |
| Liver | Healthy | 53 | (100.0%) | NA | | <.001 |
| cirrhosis | NA | NA | | 29 | (54.7%) | <.001 |
| | Yes | NA | | 24 | (45.3%) | <.001 |
| | NA | 31 | (58.5%) | NA | | <.001 |
| | Alcoholic | NA | | 3 | (5.7%) | <.001 |
| | Hepatitis | 20 | (37.7%) | 27 | (50.9%) | <.001 |
| | B | | | | | |
| | Hepatitis | NA | | 4 | (7.5%) | <.001 |
| | B + | | | | | |
| | Hepatitis | | | | | |
| | C | | | | | |
| | Hepatitis | 2 | (3.8%) | 14 | (26.4%) | <.001 |
| | C | | | | | |
| | Non-A | NA | | 5 | (9.4%) | <.001 |
| | non-B | | | | | |
| | hepatitis | | | | | |
| Age | Mean ± | 59.5 ± 9.7 | | 62.2 ± 10.4 | | 0.168 |
| | standard | | | | | |
| | deviation | | | | | |
| | Median | 59.0 | (53.0-68.0) | 62.0 | (54.0-69.0) | 0.204 |
| | (Q1-Q3) | | | | | |
| AFP | Mean ± | 5.3 ± 2.9 | | 3,445.7 ± 12,312.1 | | 0.044 |
| | standard | | | | | |
| | deviation | | | | | |
| AFP | Median | 4.4 | (3.0-6.0) | 15.6 | (6.6-294.7) | <.001 |
| | (Q1-Q3) | | | | | |

### Embodiment 2: Analysis of Microbial Flora (2)

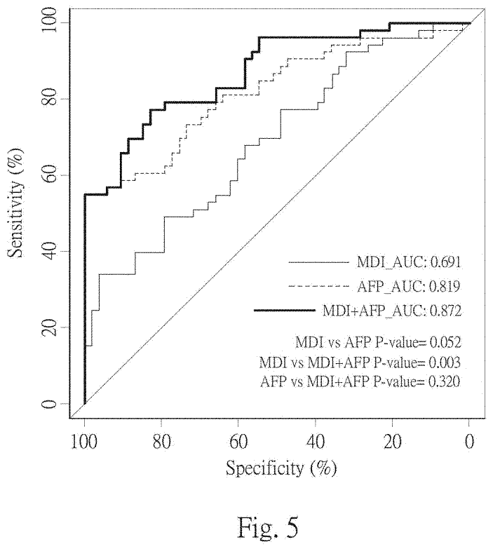

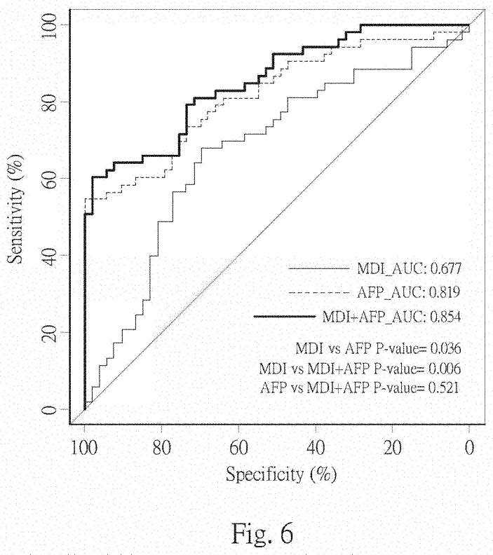

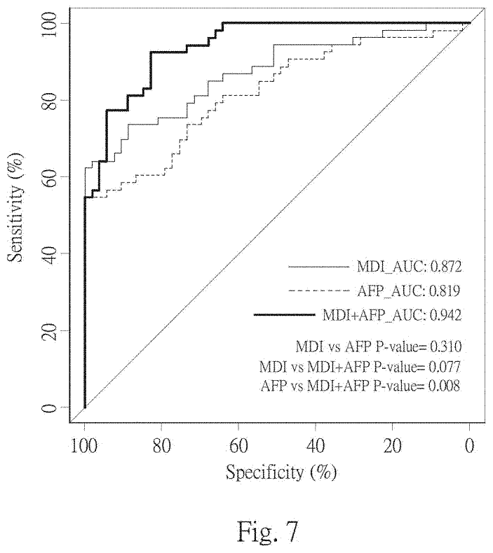

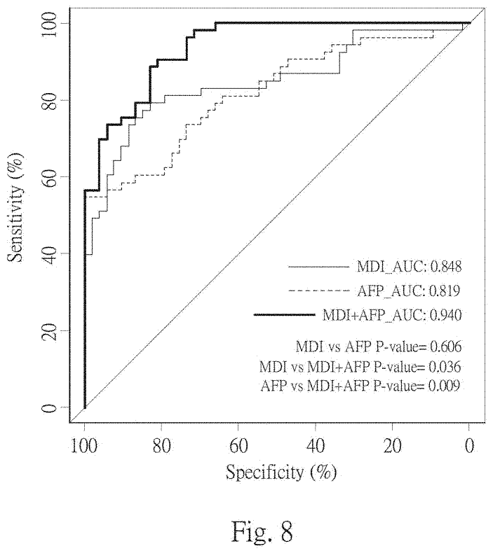

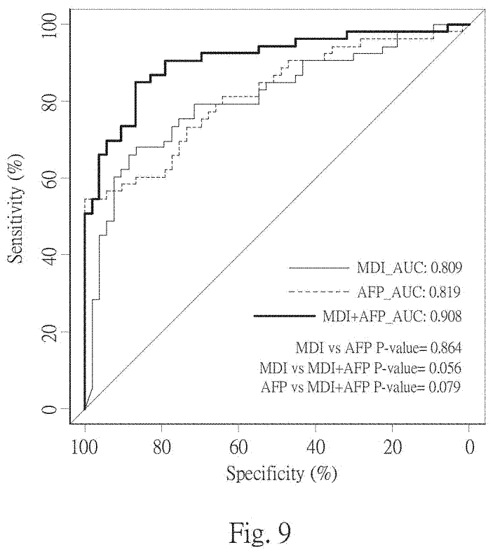

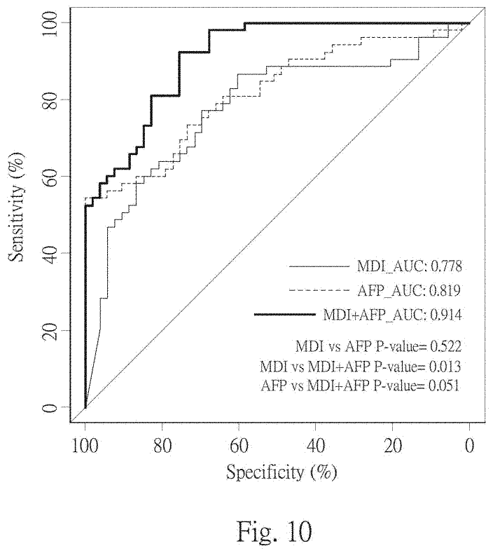

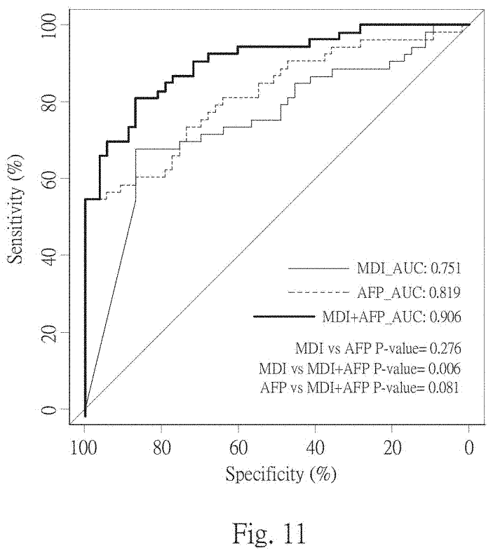

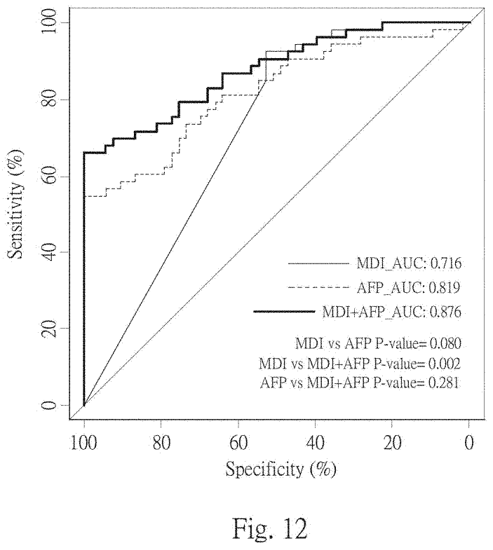

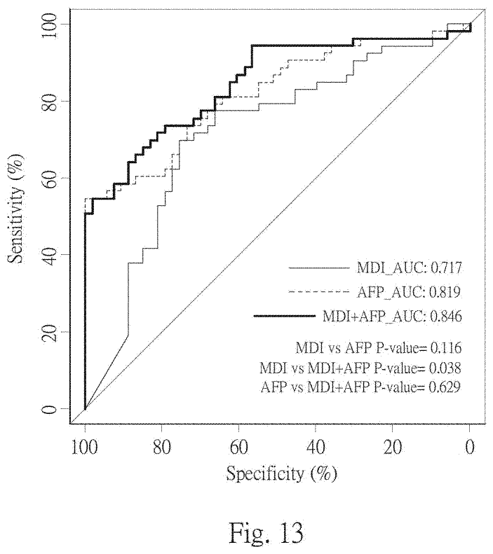

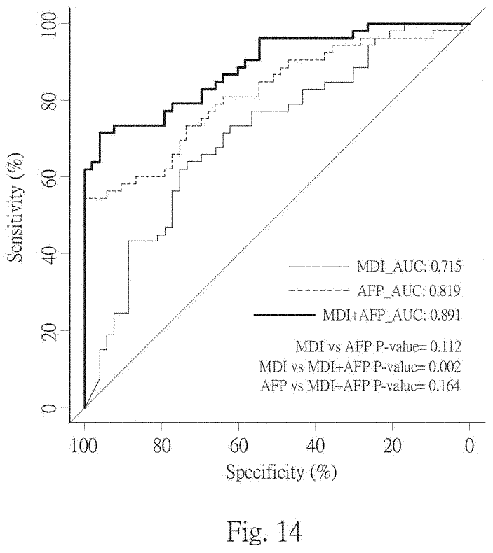

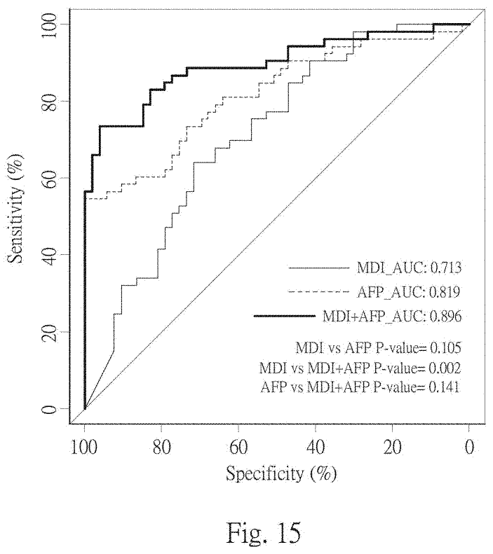

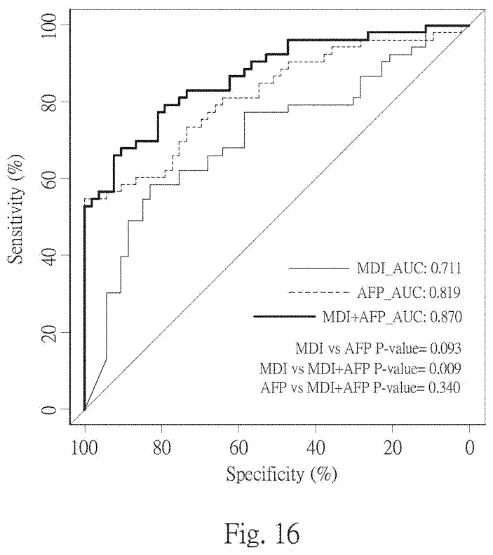

From the results obtained in embodiment 1, the bacterial genera with significant differences are selected, MDI conversion is performed respectively, alpha-fetoprotein (AFP) is combined respectively, and then ROC curve analysis is performed. The results are shown in FIGS. 5 to 16.

From the results in FIG. 5 to FIG. 16, it can be known that compared with the accuracy rate (81.9%) of using alpha-fetoprotein (AFP) alone in the diagnosis of liver cancer, the correct rate of combining alpha-fetoprotein (AFP) with different bacterial genera respectively for liver cancer diagnosis is significantly improved. Specifically, the correct rate of genus Lachnospira combined with alpha-fetoprotein (AFP) is 87.2%; the correct rate of genus Phascolarctobacterium combined with alpha-fetoprotein (AFP) is 85.4%; the correct rate of genus Megasphaera combined with alpha-fetoprotein (AFP) is 94.2%; the correct rate of genus Dialister combined with alpha-fetoprotein (AFP) is 94.0%; the correct rate of genus Streptococcus combined with alpha-fetoprotein (AFP) is 90.8%; the correct rate of genus Acidaminococcus combined with alpha-fetoprotein (AFP) is 91.4%; the correct rate of genus Lactobacillus combined with alpha-fetoprotein (AFP) is 90.6%; the correct rate of genus Enterococcus combined with alpha-fetoprotein (AFP) is 87.6%; the correct rate of genus Eubacterium combined with alpha-fetoprotein (AFP) is 84.6%; the correct rate of genus Bifidobacterium combined with alpha-fetoprotein (AFP) is 89.1%; the correct rate of genus Collinsella combined with alpha-fetoprotein (AFP) is 89.6%; and the correct rate of genus Clostridium combined with alpha-fetoprotein (AFP) is 87.0%.

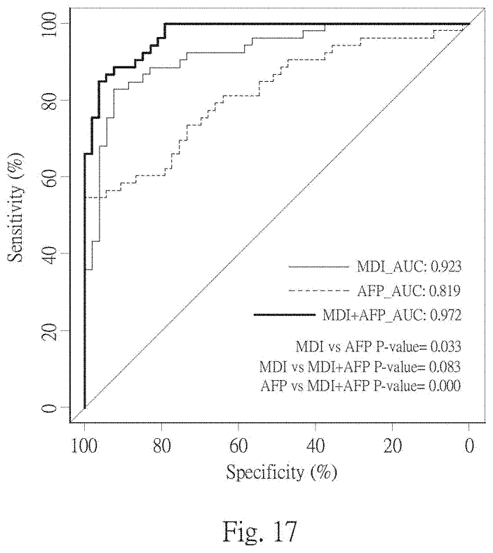

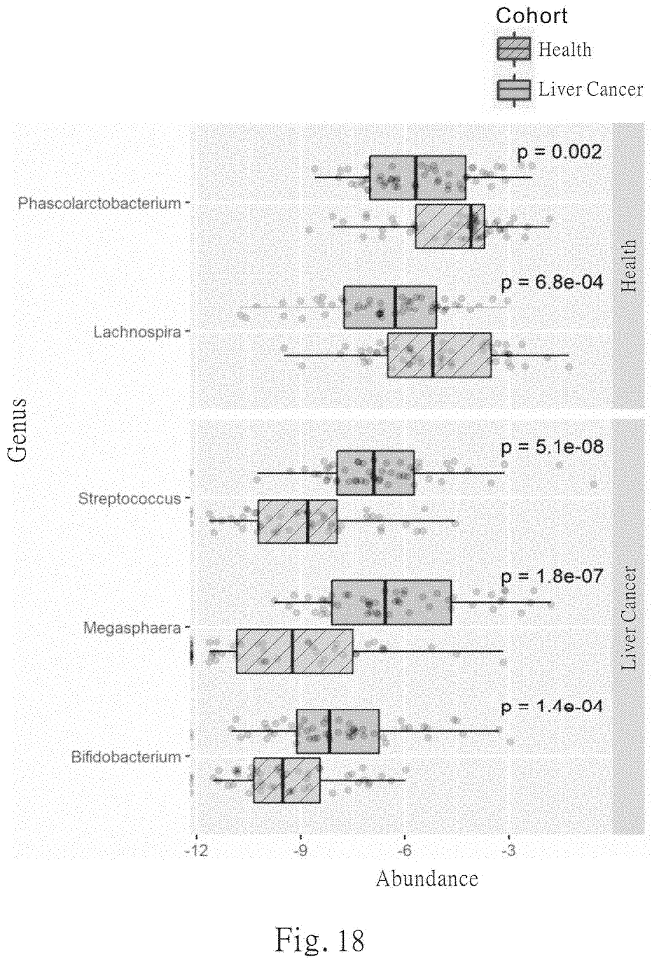

Further, five bacterial genera: Phascolarctobacterium, Lachnospira, Megasphaera, Streptococcus and Bifidobacterium are selected from the above results, MDI conversion is performed, and combined with alpha-fetoprotein (AFP) for ROC curve analysis. The results are shown in FIG. 17; and, the relative content distribution of the five bacterial genera is analyzed, as shown in FIG. 18.

From the results in FIG. 17, it can be known that compared with the accuracy rate (81.9%) of using alpha-fetoprotein (AFP) alone in the diagnosis of liver cancer, the correct rate of combining alpha-fetoprotein (AFP) with the above five bacterial genera respectively for liver cancer diagnosis is significantly improved to 97%.

### Embodiment 3: Analysis of Microbial Flora (3)

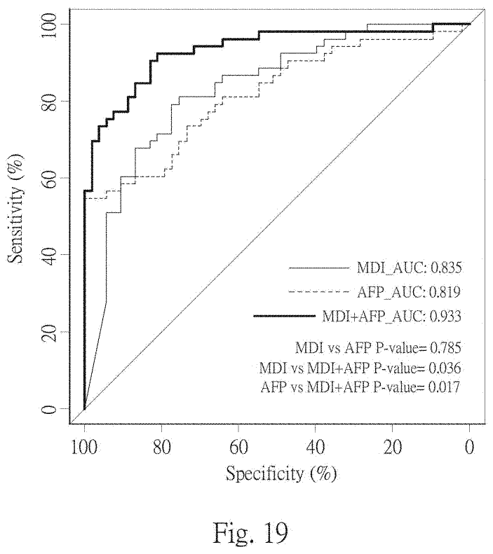

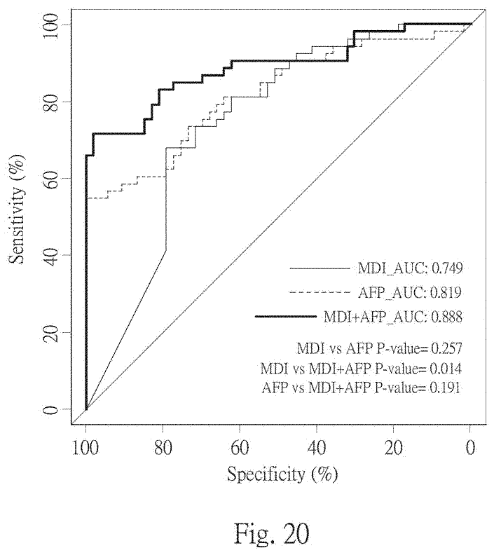

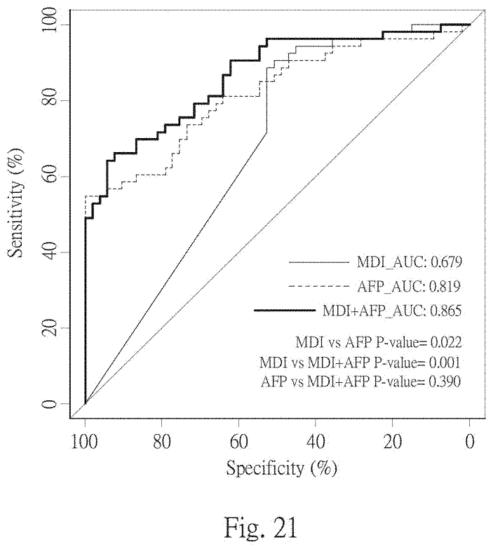

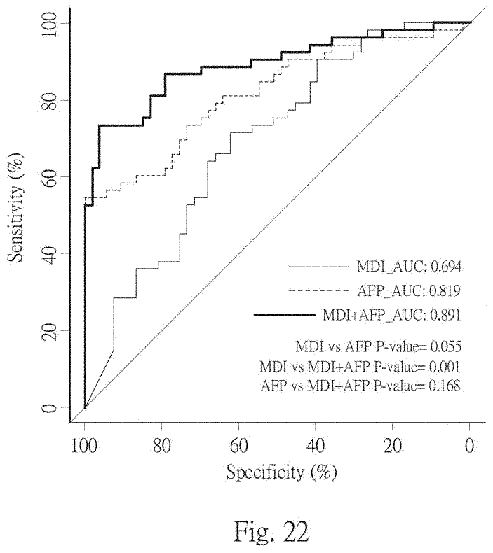

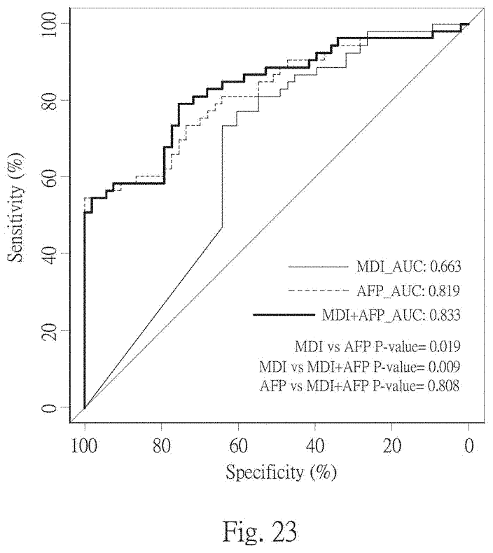

From the results obtained in embodiment 1, the bacterial species with significant differences are select, MDI conversion is performed respectively, and then combined with alpha-fetoproteins respectively to perform ROC curve analysis. The results are shown in FIGS. 19 to 23.

From the results of FIG. 19 to FIG. 23, it can be known that compared with the accuracy rate (81.9%) of using alpha-fetoprotein (AFP) alone in the diagnosis of liver cancer, the correct rate of combining alpha-fetoprotein (AFP) with different bacterial species respectively for liver cancer diagnosis is significantly improved. Specifically, the correct rate of species Ruminococcus gnavus combined with alpha-fetoprotein (AFP) is 93.3%; the correct rate of species Bifidobacterium longum combined with alpha-fetoprotein (AFP) is 88.8%; the correct rate of species Rothia mucilaginosa combined with alpha-fetoprotein (AFP) is 86.5%; the correct rate of species Collinsella aerofaciens combined with alpha-fetoprotein (AFP) is 89.1%; and the correct rate of species Eubacterium dolichum combined with alpha-fetoprotein (AFP) is 83.3%.

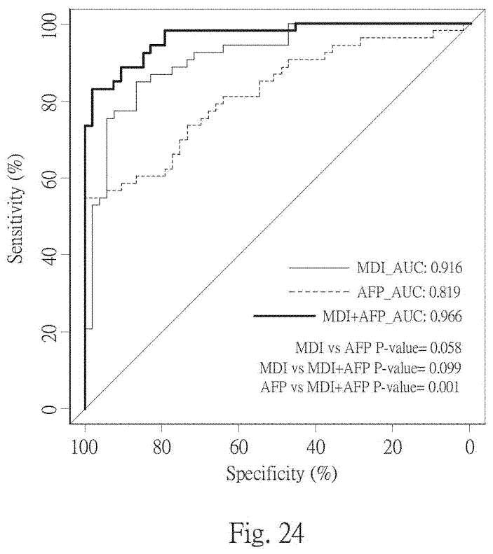

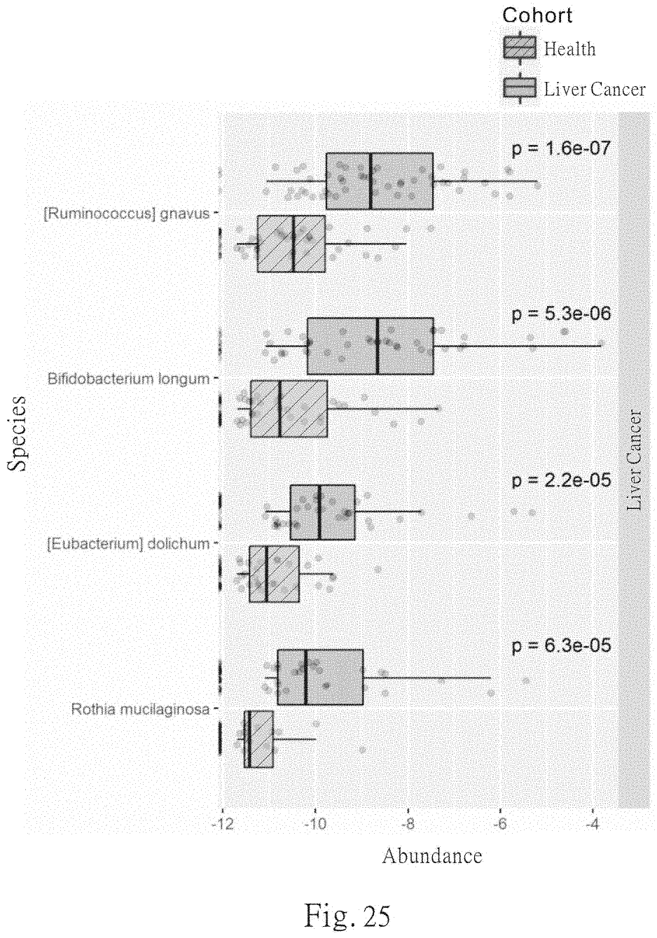

Further, Bifidobacterium longum, Ruminococcus gnavus, Eubacterium dolichum, and Rothia mucilaginosa species are selected from the above results, MDI conversion is performed, and combined with alpha-fetoprotein (AFP) for ROC curve analysis. The results are shown in FIG. 24; and, the relative content distribution of the above bacterial species is analyzed, as shown in FIG. 25.

From the results in FIG. 24, it can be known that compared with the accuracy rate (81.9%) of using alpha-fetoprotein (AFP) alone in the diagnosis of liver cancer, the correct rate of combining alpha-fetoprotein (AFP) with the above four bacterial species respectively for liver cancer diagnosis is significantly improved to 96.6%.

It is to be understood that the above description is only embodiments of the invention and is not used to limit the present invention, and changes in accordance with the concepts of the present invention may be made without departing from the spirit of the present invention, for example, the equivalent effects produced by various transformations, variations, modifications and applications made to the configurations or arrangements shall still fall within the scope covered by the appended claims of the present invention.

1-4. (canceled)

5. A biomarker for auxiliary diagnosis and detection of liver cancer, comprising at least one microorganism, wherein the microorganism comes from the following groups of bacterial genera: Lachnospira, Succinivibrio, Phascolarctobacterium, rc4_4, Sutterella, WAL 1855D, Methanobrevibacter, Bacteroides, Megasphaera, Dialister, Streptococcus, Acidaminococcus, Epulopiscium, Lactobacillus, Enterococcus, Eubacterium, Rothia, Bifidobacterium, Collinsella, Clostridium, Leuconostoc, Ruminococcus, Rhodococcus, Catenibacterium, Mitsuokella, Roseburia, Granulicatella, Citrobacter, Lactococcus, Coprobacillus, Blautia, Klebsiella, Helicobacter, Succiniclasticum, Peptococcus, Megamonas, Peptostreptococcus, Veillonella, Salmonella, Akkermansia, Corynebacterium and Faecalibacterium.

6. The biomarker for auxiliary diagnosis and detection of liver cancer as claimed in claim 5, wherein the biomarker comprises microorganisms of the following five bacterial genera: Phascolarctobacterium, Lachnospira, Megasphaera, Streptococcus and Bifidobacterium.

7. A biomarker for auxiliary diagnosis and detection of liver cancer, comprising at least one microorganism, wherein the microorganism comes from the following groups of bacterial species: Akkermansia muciniphila, Faecalibacterium prausnitzii, Bacteroides barnesiae, Eubacterium biforme, Haemophilus parainfluenzae, Veillonella dispar, Roseburia faecis, Bifidobacterium longum, Collinsella aerofaciens, Lactobacillus mucosae, Helicobacter pylori, Ruminococcus gnavus, Eubacterium dolichum, Rothia mucilaginosa, Mitsuokella multacida, Collinsella stercoris, Lactobacillus zeae, Coprobacillus cateniformis and Rothia aeria.

8. The biomarker for auxiliary diagnosis and detection of liver cancer as claimed in claim 7, wherein the biomarker comprises microorganisms of the following four bacterial species: Bifidobacterium longum, Ruminococcus gnavus, Eubacterium dolichum and Rothia mucilaginosa.

9. A method for increasing an accuracy rate of detecting liver cancer risk in vitro, comprising simultaneously detecting and analyzing manifestations of at least one biomarker as claimed in claim 5 in a stool sample and alpha-fetoprotein (AFP) content in a blood sample.

10. The method for increasing an accuracy rate of detecting liver cancer risk in vitro as claimed in claim 9, wherein the biomarker comprises microorganisms of the following five bacterial genera: Phascolarctobacterium, Lachnospira, Megasphaera, Streptococcus and Bifidobacterium.

11. A method for increasing an accuracy rate of detecting liver cancer risk in vitro, comprising simultaneously detecting and analyzing manifestations of at least one biomarker as claimed in claim 7 in a stool sample and alpha-fetoprotein (AFP) content in a blood sample.

12. The method for increasing an accuracy rate of detecting liver cancer risk in vitro as claimed in claim 11, wherein the biomarker comprises microorganisms of the following four bacterial species: Bifidobacterium longum, Ruminococcus gnavus, Eubacterium dolichum and Rothia mucilaginosa.
